(19) United States
(12) Patent Application Publication
(10) Pub. No.: US 2024/0188803 A1
(43) Pub. Date: Jun. 13, 2024

(54) ENDOSCOPE, IN PARTICULAR A MEDICAL SINGLE-USE ENDOSCOPE

(71) Applicant: KARL STORZ SE & CO. KG, Tuttlingen (DE)

(72) Inventors: Alexander FUCHS, Steißlingen (DE); Tobias LOTTES, Helmbrechts (DE)

(73) Assignee: KARL STORZ SE & CO. KG, Tuttlingen (DE)

(21) Appl. No.: 18/554,957

(22) PCT Filed: Apr. 11, 2022
(86) PCT No.: PCT/EP2022/059654

§ 371 Date: Oct. 11, 2023

(30) Foreign Application Priority Data
Apr. 12, 2021 (DE) 10 2021 109 023.7

(51) Int. Cl. A61B 1/00; A61B 1/05

(52) U.S. Cl. CPC A61B 1/00103; A61B 1/00105; A61B 1/00128; A61B 1/05

(57) ABSTRACT

Provided is a disposable medical endoscope with a handle housing for gripping and/or operating the endoscope and/or for receiving functional components. A shaft mechanically connects the handle housing to the head along a longitudinal axis and the head has an image recording means for recording an image of a viewing region. The handle housing has a connection device with a clamping element or a plurality of clamping elements and a tensioning element. The clamping element(s) allow(s) the shaft to be inserted into the connection device in a mounting position of the tensioning element. The tensioning element moves the clamping element or the clamping elements mechanically to the shaft in a fastening position, in such a way that the shaft is received in a mechanically fixed manner on the handle housing by means of a force fit or form fit between the clamping element or clamping elements and the shaft.

### CROSS-REFERENCE TO RELATED APPLICATIONS

This application is the U.S. national stage of PCT/EP2022/059654 filed on Apr. 11, 2022, which claims priority of German Patent Application No. 10 2021 109 023.7 filed on Apr. 12, 2021, the contents of which are incorporated herein.

### TECHNICAL FIELD

The disclosure relates to an endoscope system, in particular a disposable medical endoscope, with a handle housing for gripping and/or operating the endoscope and/or for receiving functional components, and with a shaft and a tip, wherein the shaft mechanically connects the handle housing to the tip along a longitudinal axis, and the tip has an image recording means for recording an image of a viewing region.

### BACKGROUND

Known endoscope systems of the type mentioned, in particular disposable medical endoscopes, are characterized by the fact that they are only suitable for one-time use and must therefore be easy and inexpensive to assemble and produce. Such a one-time use is particularly indicated when special operating conditions require rapid use of the endoscope system or even when cleaning and/or sterilizing of the endoscope system is not economical for certain procedures and/or examinations.

Hitherto, in such endoscope systems, the shaft has been connected to the handle housing, for example with an adhesive connection. This can result in overdosing or underdosing of the adhesive. In the event of overdosing, unsightly adhesive beads will appear: in the event of underdosing, a corresponding connection may not be sufficiently strong. Furthermore, adhesives are often used that harden using UV light, such that concealed regions only harden slowly and in an undefined manner after the actual production process.

### SUMMARY

It is an object of the disclosure to improve the prior art.

The object is achieved by an endoscope system, in particular a disposable medical endoscope, with a handle housing for gripping and/or operating the endoscope system and/or for receiving functional components, and with a shaft and a tip, wherein the shaft mechanically connects the handle housing to the tip along a longitudinal axis, and the tip has an image recording means for recording an image of a viewing region, wherein the handle housing has a connection device with a clamping element or a plurality of clamping elements and a tensioning element, wherein the clamping element or the clamping elements allows or allow the shaft to be inserted into the connection device in a mounting position of the tensioning element, and the tensioning element moves the clamping element or the clamping elements mechanically to the shaft in a fastening position, in such a way that the shaft is received in a mechanically fixed manner on the handle housing by means of a force fit or form fit between the clamping element or clamping elements and the shaft.

Such a connection of the shaft to the handle housing is particularly simple and can be effected, for example, without additional aids or tools and/or, for example, without adhesives. The tensioning element can be transferred from the mounting position into the fastening position, whereby the clamping element or the clamping elements are moved to the shaft, i.e. they then fix the shaft by force-fit or form-fit engagement. For example, this can be done by clamping the shaft using the clamping element or clamping elements.

The following terms are explained here:

An “endoscope system”, for example, is a device with which the interior of organisms, technical devices or other cavities can be examined and/or manipulated. For example, such an endoscope system is used for medical diagnostics. In particular, with so-called chip-on-tip endoscopes, an image recording chip attached to the shaft of an endoscope can record corresponding image information from a viewing region and, for example, forward it to a display device, such as a monitor. A “disposable medical endoscope” is explicitly designed for one-time use. Thus, such a disposable endoscope cannot and should not be cleaned, disinfected or sterilized. Such disposable endoscopes are used, for example, in emergency medicine or for special procedures and diagnoses. The “endoscope” in the narrower sense includes in particular only an imaging part such as the image recording chip and necessary mechanical components for guiding and manipulating the image recording chip and can have additional assemblies of an endoscope system, for example a handle, connections or the like. However, the terms “endoscope system” and “endoscope” and also “endoscopic device” are often also used synonymously.

A “handle housing” describes the part of the endoscope system which an operator of the endoscope system holds and, for example, operates the endoscope, triggers functions of the endoscope or also positions the endoscope using the handle housing.

“Functional components” are all components which, in addition to the shaft and the tip, i.e. the basic components of each endoscope, are arranged within the handle housing or on the handle housing. Such functional components are, for example, suction hoses, wire guides for manipulating the tip, valves or electrical assemblies or, for example, an electronic circuit board for evaluating and controlling the endoscope.

A “shaft” designates an elongate portion of the endoscope or of the endoscope system, which elongate portion connects the handle housing to the tip and thus enables the tip to be manipulated using the handle housing. Such a shaft is, for example, a tubular body, wherein both a channel for the passage of tools and, for example, electrical conductors, cable guides and the like is or are arranged within the tubular body.

A “tip” of an endoscope describes an assembly which is arranged at a front end of the shaft and in which there are arranged in particular devices for illuminating a viewing region, an image recording means, or also an output of a manipulation channel arranged in the shaft. The tip of the endoscope is guided in the direction of the viewing region by means of the shaft and, for example, inserted into the patient during an operation.

A “longitudinal axis” describes a reference axis that runs along the shaft and along the tip. Such a longitudinal axis does not have to be an axis of gravity or an axis of symmetry of the shaft and/or of the tip, but generally describes an axis that runs along the longitudinal extent of the shaft and also the longitudinal extent of the tip. Such a longitudinal axis can also be curved in the region of the tip, for example if the tip of the endoscope is designed to be pivotable or angled.

An “image recording means” describes any means that is suitable for capturing an image of the viewing region. For example, such an image recording means is an image recording chip which is arranged at the tip and which is then connected to the handle housing, and to electronic assemblies in the handle housing, by means of electrical conductors through the shaft. Furthermore, however, such an image recording means can also be a lens system, which then uses a light guide to guide a corresponding image of the viewing region through the shaft to the handle housing and, for example, to an eyepiece.

An “image” can be any pictorial representation or an electronic or other equivalent of this pictorial representation that allows conclusions to be drawn about the properties or nature of the viewing region. For example, such an image is an electronic signal generated by an image recording chip at the tip of the endoscope, which signal is then displayed again on a monitor located outside the endoscope.

A “viewing region” is the region that is to be examined, viewed or manipulated using the endoscope. For example, in the case of a human medical examination, such a viewing region is an organ, a blood vessel or a bone. If the endoscope is not used medically but in a technical field, the viewing region can be, for example, the interior of an internal combustion engine, the interior of a gear transmission, or another comparable location into which the endoscope provides a view.

A “connection device” is a mechanical component by means of which the shaft of the endoscope is connected to the handle housing of the endoscope system. In the case of the present disclosure, this connection device has a clamping element or a plurality of clamping elements. In addition, this connection device has a tensioning element, or a tensioning element is assigned to the connection device.

A “clamping element” is, for example, a tongue-like mechanical element or an element acting on the principle of a wedge, which is suitable for exerting forces in the direction of the shaft, in particular in the direction of the longitudinal axis of the shaft, and thus exerting a clamping force on the shaft.

A “tensioning element” is a technical device which is suitable for mechanically acting on a clamping element or several clamping elements such that they exert their mechanical function in the direction of the shaft. For example, such a tensioning element is designed in such a way that a plurality of clamping elements can be compressed by means of the tensioning element, such that the shaft is then clamped within the connection device in this compressed position. For example, a tensioning element is a tensioning ring, a tensioning wire or a tensioning sleeve, each of which presses or guides the clamping element or the clamping elements to the shaft.

A “mounting position” is the position of the tensioning element in which insertion of the shaft into the connection device, in particular between the clamping elements, is possible. For example, in this mounting position, the one clamping element or the plurality of clamping elements is or are relieved of stress, such that a free space or an interrupted frictional connection to the shaft is created, and the shaft can therefore be inserted into the connection device without resistance or with reduced resistance.

By contrast, a “fastening position” is the position in which the clamping element or the plurality of clamping elements is or are brought into engagement with the shaft by means of the tensioning element and/or is or are mechanically moved to the shaft, such that the shaft is mechanically fixed on the handle housing by means of a force fit or form fit between the clamping element or clamping elements and the shaft.

A “force fit”, which is also called a “frictional connection”, describes the force-fit connection of two surfaces, wherein a normal force is present between the surfaces to be connected, and a static friction present between the surfaces prevents the surfaces from moving against one another. A force fit is therefore able to withstand a force acting tangentially between the surfaces, which is less than or equal to the so-called static friction between the surfaces.

A “form fit” describes a so-called form-fit connection, wherein this form-fit connection is created by the interlocking of at least two connection partners. This means that the connection partners cannot separate from each other, by preventing each other from moving counter to the direction of force.

“Mechanically fixed” describes a state in which the shaft cannot be moved axially and/or rotationally relative to the handle housing. For example, the shaft is mechanically fixed if it is prevented from longitudinal movement in the handle housing and from rotation in the handle housing by means of a sufficiently strong frictional connection. Alternatively, the shaft is also mechanically fixed if the shaft is prevented from longitudinal movement and/or rotation in the handle housing by a form-fit engagement on the connection device.

In order to design the endoscope system, the endoscope and/or the connection device simply and effectively, the clamping element or the clamping elements is or are arranged parallel or substantially parallel to the longitudinal axis, in particular distributed around the longitudinal axis.

This means, for example, that a number of clamping elements can be arranged uniformly around the longitudinal axis, such that the shaft is mechanically fixed uniformly from all sides around the longitudinal axis. For example, the clamping element or the clamping elements is or are then designed in the form of tongues or clamping tabs running along the longitudinal axis, such that the tensioning element can be used to clamp or mechanically fix the shaft according to the wedge principle.

In one embodiment, the clamping element or clamping elements has or have a structuring facing the shaft, wherein in particular the shaft has a corresponding structuring facing the clamping element or clamping elements.

By means of such structuring, the force fit or form fit between the shaft and the connection device can be significantly increased, such that a more durable connection is created. Here, structuring can be provided only on the respective clamping element or only on the shaft, such that the structuring is completely or partially impressed on the respective other component during the assembly process. For example, a structuring in the clamping elements can act on a soft surface of the shaft facing the clamping elements in such a way that the structuring of the clamping elements is impressed into this soft surface during assembly and fastening.

A “structuring” describes any surface change of the respective surface on the respective clamping element facing the shaft and/or on the surface of the shaft facing the clamping element or clamping elements. For example, such a structuring is a roughening or texturing of the respective surface.

In order to improve a force-fit connection, the structuring and/or the corresponding structuring act or acts frictionally by means of the force fit between the clamping element or clamping elements and the shaft, such that a coefficient of friction is increased and the shaft is thus mechanically fixed in the handle housing with a higher load capacity.

The so-called static friction between the clamping element or clamping elements and the shaft can thus be increased, such that greater forces are necessary to move the shaft axially or also rotationally relative to the handle housing. Overall, this creates a more robust connection.

A “coefficient of friction” describes the factor between the normal force and the tolerable tangential force between two frictionally connected surfaces and/or also between two surfaces connected by force-fit engagement. For example, such a coefficient of friction can be increased by roughening the respective surfaces.

In one embodiment, the structuring and the corresponding structuring engage in respective corresponding structural elements on the shaft by means of respective structural elements in a form-fitting manner between the clamping element or the clamping elements and the shaft, wherein in particular structural elements and corresponding structural elements are arranged along the longitudinal axis in order to increase a transmissible torque between the shaft and the connection device and/or structural elements and corresponding structural elements are arranged along a circumference around the longitudinal axis in order to increase transmissible tensile forces.

A clear or unique mechanical connection can thus be created for the shaft in the connection device. Such a unique arrangement of the shaft can be effected, for example, by means of structural elements which are arranged according to the key and lock principle and only allow a single rotational and axial arrangement. A specific mounting position can thus be predetermined. Arranging structural elements and corresponding structural elements along the longitudinal axis creates a rotational connection, analogous to a tooth connection, while arranging structural elements and corresponding structural elements along a circumference around the longitudinal axis creates a form-fit connection against tensile forces.

Furthermore, the respective structural elements can also be provided only on the respective clamping element or only on the shaft, such that the corresponding structural element is completely or partially impressed on the respective other component during the mounting process. For example, a structural element in the clamping elements can act on a soft surface of the shaft facing the clamping elements, in such a way that the structuring of the clamping elements is impressed into this soft surface during the mounting and fastening

A “structural element” describes a deliberately generated and geometrically defined surface change of a clamping element and/or of the shaft, in such a way that the respective structural element and a respective corresponding structural element interlock with each other in a form-fitting manner. For example, such a structural element is a projection, whereas a structural element corresponding thereto is a depression formed inversely thereto.

In order also to be able to transfer a plurality of clamping elements, or a clamping element arranged rotationally around the shaft, safely into the fastening position, the tensioning element is annular and the clamping element or clamping elements are partially or completely surrounded by the tensioning element in the fastening position.

Such a tensioning element can, for example, be realized in a ring shape in the form of a conically tapering tubular component, so that such a conically tapering tensioning element tensions corresponding conical outer surfaces of the clamping element or of the respective clamping elements according to the principle of a wedge, when transferred into the fastening position along the longitudinal axis, and thus mechanically fixes the shaft.

In one embodiment, the tensioning element has a latch or a plurality of latches, wherein the latch or the latches engages or engage in the fastening position in a corresponding latch receptacle or a respective corresponding latch receptacle on the connection device, wherein the latch or the latches are designed and mechanically weakened in particular by means of a weakening, in such a way that a movement of the tensioning element out of the fastening position causes the respective latch and/or the respective latch receptacle to be destroyed.

Thus, the tensioning element can be fixed in the fastening position, in particular without tools, by locking the latches to the respective corresponding latch receptacle. In particular, by means of the weakening, repeated separation and/or joining of the connection achieved can be prevented, such that attempts at manipulation by an operator lead to the destruction of the endoscope. This creates certainty, particularly in the case of disposable medical endoscopes, that repeated use after manipulation is ruled out.

A “latch” and a corresponding “latch receptacle” are corresponding components that establish a form-fit connection to each other by means of latching. For example, this is a connection consisting of a latching lug as latch receptacle and of a correspondingly shaped hook or a corresponding eyelet as latch, wherein the hook and/or the eyelet then slide over the latching lug and lock onto it. In particular, such an arrangement of latch and latch receptacle is designed according to the principle of a barb, such that joining together is easy, but loosening is effectively prevented.

A “weakening” is, for example, a mechanical constriction, a material slimming or targeted previous damage to a corresponding part of the latch receptacle and/or of the latch, which results in a one-time locking of the respective latch onto the respective latch receptacle being mechanically tolerated, but a separation of the latching connection, in particular counter to a latching direction, leads to the destruction of the respective latch and/or of the respective latch receptacle.

In order to be able to easily bring the tensioning element into engagement by sliding it over the shaft and thus to be able to mount the endoscope easily, the latch is designed to act axially along the longitudinal axis, such that the tensioning element is movable from the mounting position into the fastening position along the longitudinal axis and is fixed in the fastening position in a latching manner by means of the latch or latches.

In one embodiment, the latch or the latches forms or form a bayonet lock, wherein the latch receptacle or the latch receptacles is or are designed in particular as latching lugs protruding from the connection device, and the latch or the latches is or are designed to be hook-shaped, such that the tensioning element can be moved along the longitudinal axis into the fastening position and can be fixed in the fastening position by means of a rotation about the longitudinal axis.

Such a bayonet lock can be used additionally or as an alternative, such that the particularly annular tensioning element is first pushed over the shaft of the endoscope, then the tensioning element is brought into the fastening position, the clamping element or clamping elements being brought into operation, and then the tensioning element being accordingly fixed by means of a rotation about the longitudinal axis. The bayonet lock can also be designed in such a way that loosening of the tensioning element from the fastening position leads to the destruction of the connection. This is effected by a weakening, analogous to the above example, of corresponding components of the bayonet lock.

In order to enable reversible mechanical fixing of the shaft to the handle housing, the tensioning element can be screwed onto the connection means by means of a thread, such that the tensioning element is screwed on in the fastening position.

A reversible or partially reversible connection can thus be created, wherein the tensioning element can also be fixed to the connection means by means of an additional adhesive or another fixing device, such that the connection designed as a thread can also be made irreversible.

In a further embodiment, the tensioning element, in particular on an accessible outer side facing away from the shaft, has a structure for applying forces for transferring the tensioning element into the fastening position, such that the forces for transferring the tensioning element into the fastening position can be applied manually by means of the structure and/or by means of a tool.

By means of this structure, corresponding forces can then be applied to the tensioning element manually or also using a tool, such that transfer to the fastening position is considerably easier.

A “structure” is, for example, a fluting, a uniform or non-uniform arrangement of pockets, projections or recesses, or any other geometric change to a corresponding surface, which increase friction with, for example, the fingers of an operator or also permit a force-fit or form-fit connection with a corresponding tool.

In order to permit radial engagement by means of fingers or a tool, the structure has depressions introduced radially into the tensioning element or elevations applied to the tensioning element, and/or engagement parts for the fingers of an operator and/or for a corresponding tool which are introduced axially into the tensioning element or applied to the tensioning element, in particular surrounding the shaft.

By means of such depressions and/or elevations or also engagement parts, a structure can be applied easily and economically, particularly for a radial operating position.

A “depression” is a reduction of a surface, in the form of an impression in the surface, compared to a surrounding surface. Accordingly, an “elevation” entails a part of a surface leading or projecting out from the surface.

An “engagement part” for the fingers of an operator and/or for a tool corresponding to the engagement part is, for example, a depression and/or also an elevation, which is formed and/or adapted in particular to a finger of an operator or also to a corresponding tool.

In a further embodiment, the connection means is designed as a separate transition piece between the handle housing and the shaft and in particular has an attachment, wherein the attachment is able to be brought into engagement with a corresponding receptacle on the handle housing.

Such a separate connection means can be designed as an adapted transition or as an adapted transition piece between the handle housing and the shaft, in such a way that, for example, different handle housings can be connected to different shafts. A side of the transition piece facing the handle housing can thus be designed to match the respective handle housing and, analogously, a side of the transition piece facing the shaft can be designed to match a respective shaft. This means that a corresponding number of shafts can be combined with a corresponding number of handle housings without changing the handle housing. In particular, an attachment is arranged on the connection means, which attachment can be brought into engagement with a corresponding receptacle on the handle housing, such that mounting is easily possible, for example, by inserting the attachment into the corresponding receptacle on the handle housing. In particular, the handle housing, handle shells, the connection means and/or other constituent parts of the endoscope system are manufactured by injection molding. This enables economical production of the corresponding components.

A “transition piece” is, for example, a component which is designed on a first side to correspond to the handle housing and is designed on a second side to correspond to the shaft. A transition piece is in particular designed in one piece and can be realized, for example, as a simple injection-molded part and at the same time can include the clamping element or the clamping elements.

An “attachment” is, for example, a geometric configuration of the side of the transition piece facing the handle housing, which can, for example, establish a form-fit connection with a receptacle on the handle housing corresponding to the attachment.

In order to considerably simplify mounting of the endoscope and, for example, to be able to manipulate cables guided within the shaft, or printed conductor elements guided within the shaft, manually at the end of the shaft in such a way that connection to other functional components within the handle housing is possible, the connection means has a passage arranged orthogonally or substantially orthogonally to the longitudinal axis in order to facilitate mounting of the shaft on the handle housing, such that connections for the image recording means and/or mechanical control means guided within the shaft and protruding beyond one end of the shaft can be reached by means of a finger or several fingers of an operator, by a tool such as tweezers, for example, or also by means of another aid. A tool such as tweezers or pliers offers additional safety in relation to the sterility and/or cleanliness of an assembly. In particular, the passage is realized in the region of the attachment or in the attachment.

“Orthogonal” or “substantially orthogonal” describes a direction that is arranged orthogonally to the longitudinal axis within technical limits. For example, a deviation of between 30° and −30° in relation to a mathematical orthogonality to the longitudinal axis is permissible. What is particularly important here is that lateral access, for example to connections for the image recording means and/or mechanical control means protruding beyond one end of the shaft, is made possible by means of a finger or several fingers of an operator, in particular in the region of the attachment of the connection means. The corresponding connection means and/or the corresponding separate transition piece can thus be made as short as possible with respect to the longitudinal axis. Angle details refer here to a full angle of 360°.

A “passage” is a mechanical cutting or a targeted omission of material, for example in an order of size that corresponds geometrically to the cross section of a finger or several fingers of an operator.

“Connections for the imaging means” and/or “mechanical control means” are all components that are routed through the shaft or within the shaft and that connect the tip to further technical devices within the handle housing. For example, such connections for the image recording means are printed conductor tracks on a corresponding backing paper. Mechanical control means can be, for example, steering wires for the targeted manipulation of the tip of the endoscope. Using such steering wires, the tip of the endoscope can then be pivoted, for example, so that an angle of observation of the viewing region is changed.

In a further embodiment, the handle housing has a first handle shell and a second handle shell, such that, when the first handle shell is joined to the second handle shell, the connection means designed as a transition piece is enclosed in a form-fitting manner between the first handle shell and the second handle shell. The form-fit enclosure is effected in particular by means of the attachment on the connection means.

The endoscope system can thus be mounted with two handle shells, with all functional components being inserted in particular into one handle shell. The connection means, designed as a separate transition piece, is then inserted with the previously attached shaft of the endoscope. The tensioning element can already be in the fastening position or can also be moved to this position after the shaft with the connection means has been mounted on the handle shell. The second handle shell is then placed on top, whereby the connection means is fixed to the shaft within the handle housing and the endoscope system is fully assembled. When the first handle shell is thus joined to the second handle shell, the passage can then be used to grip corresponding connections for the image recording means and/or mechanical control means using a finger or several fingers or also using a tool, for example tweezers, and to connect them to functional components within the respective handle shell. In particular, the handle shells lock by means of appropriate latching aids, so that in particular an irreversible latching connection is created. This means that the complete endoscope system is protected against unauthorized manipulation, for example by unauthorized disassembly.

The disclosure is explained in more detail using exemplary embodiments. In the drawing:

### BRIEF DESCRIPTION OF DRAWINGS

FIG. 1 shows a schematic representation of a disposable medical endoscope in a side view,

FIG. 2 shows a partially sectioned, enlarged view of a handle body of the endoscope of FIG. 1,

FIG. 3 shows a schematic representation of a transition piece for the disposable medical endoscope in an isometric view,

FIG. 4 shows an isometric view of the transition piece of FIG. 3 with a shaft mounted therein and with a tensioning sleeve,

FIGS. 5a and 5b show schematic representations of a transfer of the tensioning sleeve from a mounting position to a fastening position by means of a bayonet lock,

FIG. 6 shows an alternative tensioning sleeve in an isometric view,

FIG. 7 shows a further alternative tensioning sleeve in an isometric view,

FIG. 8 shows an assembly consisting of a transition piece and of a tensioning sleeve and a shaft using a thread,

FIG. 9 shows a transition piece with transverse webs and longitudinal webs on an inner surface for receiving a shaft in a form-fitting manner,

FIG. 10 shows an isometric representation of an alternative transition piece, and

FIG. 11 shows a tensioning sleeve corresponding to the alternative transition piece of FIG. 10.

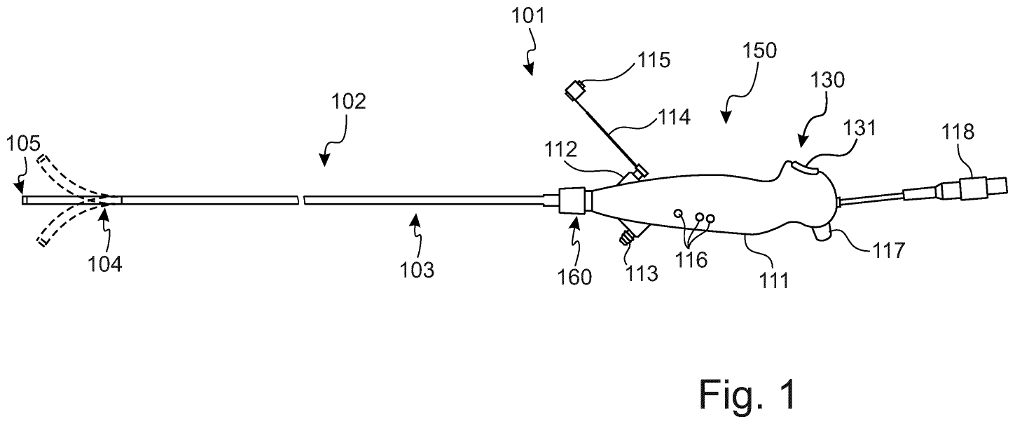

### DETAILED DESCRIPTION OF PREFERRED EMBODIMENTS

An endoscope system designed as a disposable endoscope 101 has a shaft 102 and a handle body 150. A distal shaft portion 104 is equipped with a movable camera tip 105. A proximal shaft portion 103 is fastened to the handle body 150 by means of a shaft connector 160. The disposable endoscope 101 is what is called a flexible endoscope, where the shaft 102 is flexible and can therefore follow an examined topography. Only the camera tip 105 is designed to be actively movable.

The handle body 150 has a housing 111, which is constructed from two half-shells manufactured by injection molding. An access port 112 is arranged on the housing 111. A tool can be inserted into the access port 112, which tool can then be guided through a channel (not shown) arranged in the shaft 102 as far as the camera tip 105. Such a tool can then be used to manipulate a viewing region observed with the disposable endoscope 101. In an alternative, the disposable endoscope 101 can also be implemented without the access port 112 and without a channel arranged in the shaft 102.

A closure cap 115 is attached to the access port 112 by means of a tab 114. By means of the closure cap 115, the access port 112 can be closed when not in use. The tab 114 always holds the closure cap 115 securely on the disposable endoscope 101, such that the closure cap 115 cannot be lost.

Furthermore, a hose connector 113 is arranged on the housing 111 and is connected laterally to the access port 112 by means of a hose 121 via a valve 130 and another hose 120. When the valve 130 is in the appropriate position, liquid for example can be sucked from the camera tip 105 through the channel arranged in the shaft 102 and via the hose connector 113. For this purpose, the closure cap 115 is then fitted on the access port 112 so that the latter is tight. In a further or additional alternative, the disposable endoscope 101 can also be designed without the hose connector 113 and the associated components.

The valve 130 has a push button 131 and a valve body 132. The valve 130 can be activated or deactivated using the push button 131, so that switching between a closed state and an open state is possible. This means that suction can be specifically activated by an operator by pressing the push button 131. The function of the valve is not described in any more detail here.

Operating elements 116 are also arranged on the housing 111, by means of which, for example, lighting functions, imaging functions or other functions of the disposable endoscope 101 can be controlled. In an alternative, the disposable endoscope 101 can also be designed without these operating elements.

Furthermore, at a proximal end of the housing 111, a control wheel 122 is arranged in the housing 111 and can be operated by means of a rotary knob 117 arranged outside the housing. By means of the control wheel 122, the camera tip 105 in the present example can be adjusted in one plane via a pull wire 123 and a pull wire 124, which are guided through a guide body 127 within the housing 111, such that a viewing region for the camera tip 105 can be changed or adjusted.

All functional components, such as the control wheel 122, the pull wires 123 and 124, the guide body 127, the valve 130, and also electrical and/or electronic elements (not shown), are accommodated within the housing 111. This also includes, for example, an electronic circuit board (not shown), which is arranged below the operating elements 116 within the housing 111.

The camera tip 105 of the endoscope 101 has, in addition to an imaging chip (not shown), further electronic elements for illuminating the viewing region (not shown), and an opening, facing the viewing region, for the channel (likewise not shown) arranged in the shaft 102 and leading to the housing 111. In the alternative embodiment without a channel in the shaft 102, this opening would not be present either. The disposable endoscope 101 is therefore designed as what is called a “chip-on-tip” endoscope. For this purpose, an electrical connection 118 is arranged at a proximal end of the housing 111 and serves to connect the disposable endoscope 101 to an external display unit.

The shaft 102 is received with its proximal end region 103 in the shaft connector 160 as follows:

The shaft connector 160 has a central connection piece 161, which is received in a form-fitting manner in the housing 111 by means of a constriction 167 and associated collars 169 and 170. For this purpose, the double-shell housing 111 is assembled around the constriction 167, and the connection piece 161 is received and fixed in a form-fitting manner between the half-shells of the housing 111.

The connection piece 161 also has clamping tabs 165 which are arranged symmetrically about a longitudinal axis 191 and which are each separated from one another by means of slots 173. The clamping tabs 165 are arranged symmetrically about a longitudinal axis 191. An incision 166, which runs around the circumference of the connection piece 161, mechanically separates the clamping tabs 165 from the base body of the connection piece 161 in such a way that elastic bending of the clamping tabs 165 in relation to a base body of the connection piece 161 is possible. The base body is formed in particular of the constriction 167, the collar 169, the collar 171 and a middle piece 176.

The shaft 102 is inserted with its distal end 103 along the longitudinal axis 191 into the connection piece 161 and slides along an inner side 174 of the respective clamping tabs 165 into the connection piece 161. The diameter within the inner sides 174 on the connection piece 161 is chosen such that the shaft 102 slides with its distal end 103 into the connection piece 161 without force, but with as little play as possible.

On the outer circumference of the middle piece 176, a respective latching lug 177 is also arranged at two opposite points (opposite in relation to the longitudinal axis 191). This respective latching lug 177 protrudes from the cylindrical surface of the middle piece 176 and serves to lock a tensioning sleeve 181 in a fastening position.

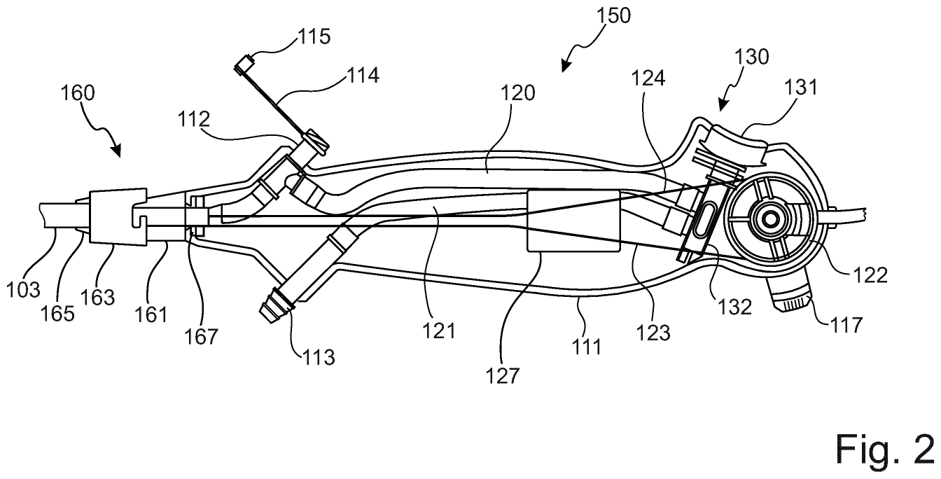

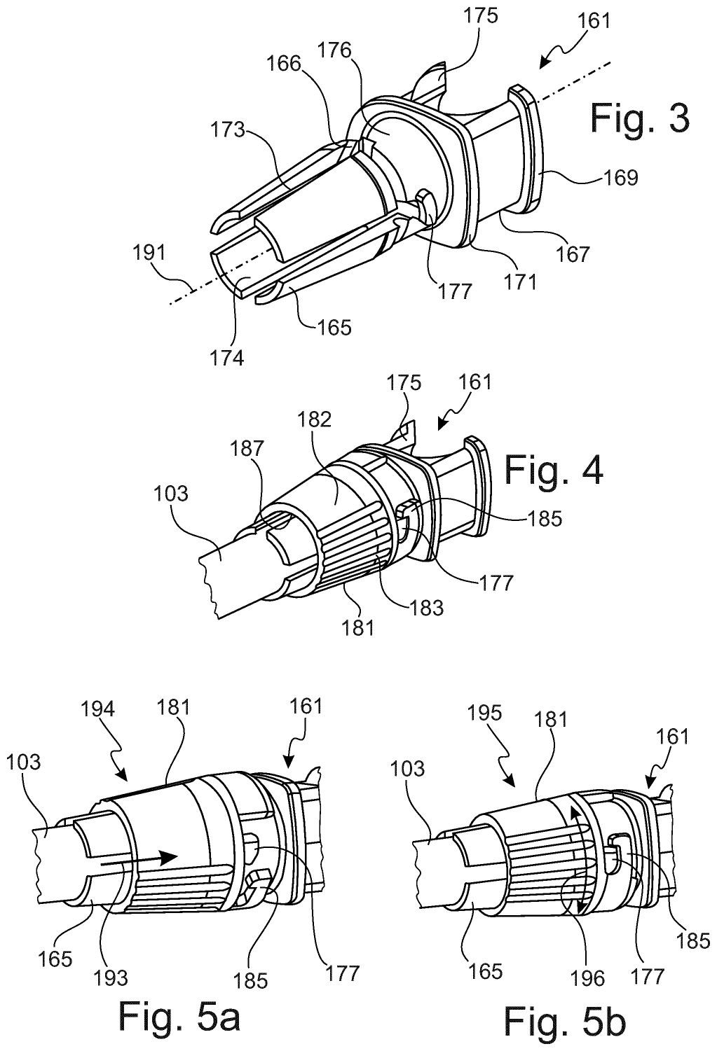

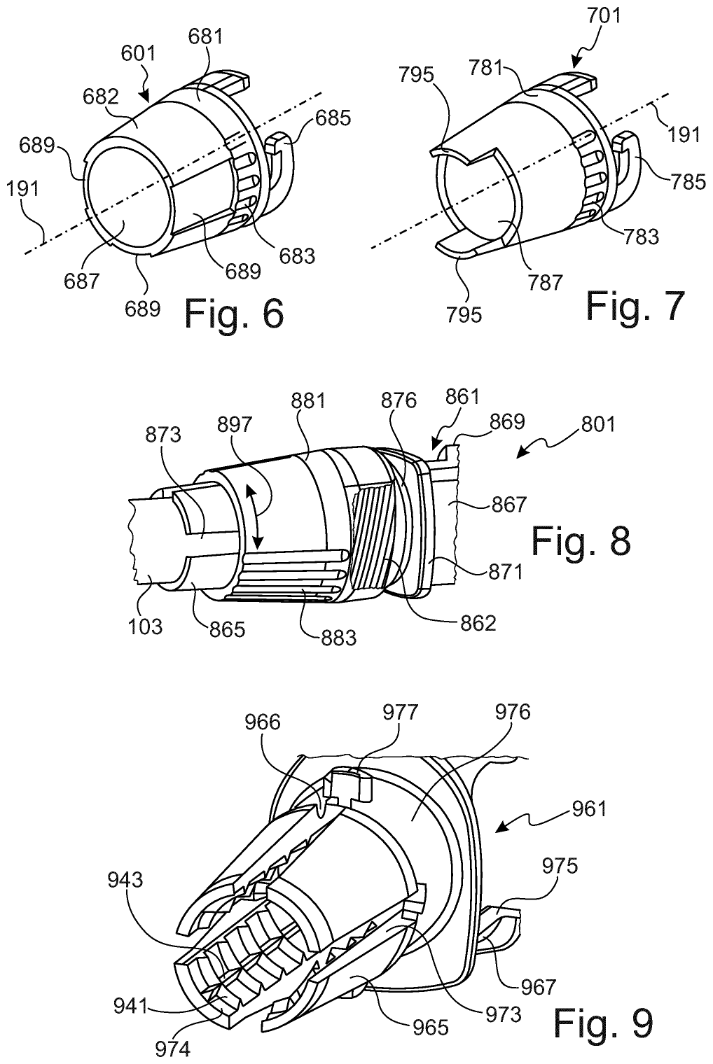

In the fastened state, the tensioning sleeve 181 is applied so completely onto the clamping tabs 165 that these clamping tabs 165 are pressed toward the distal end region 103 of the shaft 102, and the shaft 102 is thus held firmly within the connection piece 161. In the fully mounted state (see FIG. 2), the shaft 102 is thus received and fixed for conjoint rotation and displacement relative to the housing 111 by means of the connection piece 161.

The tensioning sleeve 181 is slightly conical and substantially cylindrical and has a fluting 183 on an outer surface 182. By means of the fluting 183, which runs approximately parallel to the longitudinal axis 191, the tensioning sleeve 181 can be operated manually and, in particular, can be rotated with improved force transmission.

An inner surface 187 of the tensioning sleeve 181 is shaped to correspond to the respective outer surface of the respective clamping tabs 165, such that a contact is achieved here that is as flat and precise as possible.

At a proximal end region, the tensioning sleeve 181 has hooks 185 which correspond to the latching lugs 177 and are each arranged opposite symmetrically to the longitudinal axis 191 and are designed in such a way that they engage around the latching lugs 177 according to the principle of a bayonet lock and thus fix the tensioning sleeve 181 relative to the connection piece 161.

The transfer of the tensioning sleeve 181 from a mounting position 194 to a fastening position 195 will be described again as follows (see FIG. 5a and FIG. 5b):

The tensioning sleeve 181 is first of all pushed slightly onto the connection piece 161 along a joining direction 193. The proximal end region of the shaft 102 can then be inserted into the connection piece 161 along the longitudinal axis 191. Any cables, printed conductor tracks or even contact regions of a respective control wire (not shown) that protrude from the proximal end region 103 of the shaft 102 can then be treated manually through an opening 175 provided for this purpose and can be prepared for further connection in the housing 111.

The opening 175 runs within the connection piece 161 substantially orthogonally to the longitudinal axis 191, so that it is possible to act on the connection piece 161 at this location, for example by an operator using two fingers. This makes it particularly easy to grip any material that is to be gripped, such as cable connections, printed circuit elements or also fastening regions of control wires.

The unit formed of shaft 102 and connection piece 161 is then inserted into a housing shell of the housing 111. Before this or thereafter, the tensioning sleeve 181 is pushed completely onto the connection piece 161 along the joining direction 193 until it bears on the respective latching lug 177. The clamping tabs 165 are pressed via the inner surface 187 of the tensioning sleeve 181 against the proximal end region 103 of the shaft 102, and the shaft 102 is thus fixed in the connection piece 161. The tensioning sleeve 181 is then rotated about the longitudinal axis 191 along a direction of rotation 196, so that the hooks 185 engage behind the respective latching lugs 177 and lock irreversibly. For this purpose, the hooks 185 are designed according to the principle of a barb.

Furthermore, the transitions from the tensioning sleeve 181 to the hook 185 are dimensioned such that, if an attempt were made to remove the tensioning sleeve 181 counter to the direction of rotation 196, the respective hooks 185 would break off from the tensioning sleeve 181. This effectively counteracts manipulation of the disposable endoscope 101. In an alternative, however, these transitions can also be designed to be permanent, so that subsequent manipulation is possible.

An alternative tensioning sleeve 601 is constructed substantially analogously to the tensioning sleeve 181. On its outer surface 681, the tensioning sleeve 601 likewise has a fluting 683, which simplifies manual operation and manipulation of the tensioning sleeve 601. Furthermore, hooks 685 are arranged at a proximal end of the tensioning sleeve 601, analogously to the hooks 185 of the tensioning sleeve 181. The tensioning sleeve 601 is therefore compatible with the connection piece 161 and the function is identical.

Furthermore, impressions 689 are arranged on the slightly conical outer surface 682 and provide an engagement for a corresponding tool. An inner surface 687 of the tensioning sleeve 601 acts on the clamping tabs 165 in a similar way to the inner side 187 of the tensioning sleeve 181.

A further alternative tensioning sleeve 701 likewise has a fluting 783 on an outer surface 781. Likewise, the proximal structure of the tensioning sleeve 701 with hooks 785 is analogous to the two previously described tensioning sleeves 181 and 601. The tensioning sleeve 701 is therefore likewise compatible with the connection piece 161.

However, at a distal end, the tensioning sleeve 701 has projections 795, which are arranged in a pair symmetrically to the longitudinal axis 191. These projections 795, analogously to the impressions 689, serve to provide engagement for a corresponding tool. In the case of the tensioning sleeve 701, this tool corresponding to the projections 795 can be applied axially and brought into action. An inner surface 787 acts, analogously to the previously described tensioning sleeves 181 and 601, on the clamping tabs 165 on the connection piece 161.

An alternative assembly 801 between a connection piece 861 works on the principle of a thread. The connection piece 861 has, instead of the latching hooks 177, a thread 862 on the outer side of a middle piece 876. Analogously to the connection piece 161, the connection piece 861 has a constriction 867 and also a collar 869 and a collar 871, such that the connection piece 861 is likewise compatible with the housing 111. Likewise, the connection piece 861 also has clamping tabs 865, which are separated from one another by means of slots 873 and act on the shaft 102 analogously to the previous example.

A tensioning sleeve 881 applied to the connection piece 861 has a fluting 863, such that the tensioning sleeve 881 can be rotated manually. An internal thread corresponding to the thread 862 is arranged on an inner side of the tensioning sleeve 881 (not shown), such that the tensioning sleeve 881 can be screwed onto the thread 862. This happens along a screwing movement 897. This means that the tensioning sleeve 881 can be screwed onto the connection piece 861 in such a way that the clamping tabs 865 are compressed in the direction of the proximal end region 103 of the shaft 102 and thus fix the shaft 102. This embodiment with the thread 862 is detachable. In an alternative, however, the thread 862 can also be secured with an adhesive, such that an irreversible connection is created.

A further alternative connection piece 961 is constructed at a proximal end analogously to the previous connection pieces and can be inserted in a form-fitting manner into the housing 111 with a constriction 967. Furthermore, the connection piece 961 has an opening 975, which serves to allow connections, for example, to be manipulated using the fingers. The structure of these regions of the connection piece 961 is analogous to the previously described connection pieces. Furthermore, the connection piece 961 has latching lugs 977 in a central region 976 analogously to the latching lugs 177, such that the previously described tensioning sleeves 181, 601 and 701 are compatible with this connection piece 961.

Clamping tabs 965 are separated from one another by means of slots 973 and, analogously to the previously described connection pieces, are arranged symmetrically to one another, such that a shaft can be enclosed between the clamping tabs 965. In contrast to the previously described examples, each clamping tab 965 has transverse webs 941 and longitudinal webs 943 on a respective inner surface 974. An alternative shaft (not shown) which is shaped to correspond to this connection piece 961 and to the transverse webs 941 and the longitudinal webs 943 has impressions corresponding to the transverse webs 941 and longitudinal webs 943. In an alternative, a shaft 102 with a smooth and soft surface is used, into which the transverse webs 941 and longitudinal webs 943 are impressed.

The clamping tabs 965 are elastically connected to the middle piece 976 by means of a circumferential incision 966, such that they can be elastically expanded and compressed.

For mounting, the alternative shaft matching the connection piece 961 is then inserted into the connection piece 961, with the clamping tabs 965 being pressed slightly in the direction of a larger radius. The transverse webs 941 and longitudinal webs 943 then latch into the corresponding depressions in the alternative shaft, such that the shaft is inserted in a pre-fixed position. Then, for example by means of the opening 975, connections, control wires or other elements protruding proximally from the alternative shaft can be worked on or prepared for a further assembly step. The compression of the clamping tabs 965, for finally fixing the alternative shaft in the connection piece 961, is then effected again using one of the tensioning sleeves 181, 601 or 701, which are pushed onto the connection piece 961 according to the principle of a bayonet lock and then latch irreversibly with the respective hooks 185, 685 or 785 on the latching lugs 977.

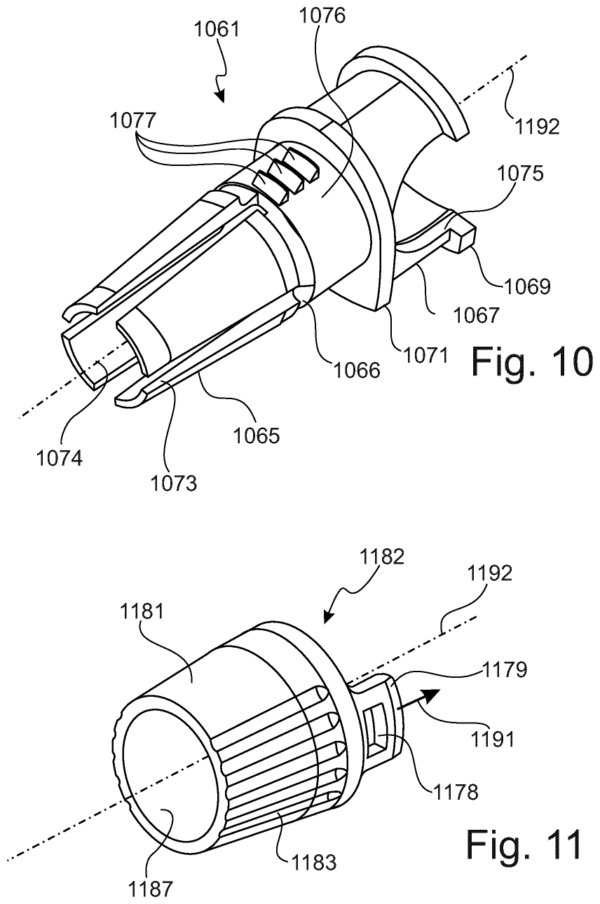

A further alternative fastening of a tensioning sleeve with a connection piece is described below (see FIG. 10 and FIG. 11):

A connection piece 1061 is constructed to be compatible with the housing 111, analogously to the previously described examples. For this purpose, the connection piece 1061 has a constriction 1067 and also a collar 1069 and a collar 1071. Likewise, an opening 1075 for an operator's fingers is arranged analogously to the previously described openings 175 and 975.

The connection piece 1061 is designed by means of clamping tabs 1065, which are separated from one another with slots 1073 and are elastically connected to a middle part 1076 by means of a circumferential incision 1066. Correspondingly smooth inner surfaces 1074 can receive the shaft 102 in a frictional manner. The clamping tabs 1065 are compressed by means of a tensioning sleeve 1181. The principle for fixing the shaft 102 is therefore designed analogously to the previously described principle for fixing to the connection piece 161.

The tensioning sleeve 1181 has a fluting 1183 on an outer surface 1182, analogously to the previously described examples, which fluting is routed approximately parallel to a longitudinal axis 1192. With an inner surface 1187, when the tensioning sleeve 1181 is pushed on along a thrust direction 1191, the clamping tabs 1065 can be compressed and the shaft 102 thus fixed.

However, in contrast to the previous embodiments, the tensioning sleeve 1181 has latching hooks 1179 at a proximal end region, each of which latching hooks 1179 has a hole 1178. Corresponding latching lugs 1077 are arranged along the longitudinal axis 1192 on the connection piece 1061. The tensioning sleeve 1181 can thus be pushed onto the connection piece 1061 along the longitudinal axis 1192 in the thrust direction 1191. The latching hooks 1179 then lock onto the holes 1178 via a respective latching lug 1077, such that the tensioning sleeve 1182 is irreversibly fixed on the connection piece 1061. The latching hooks 1179 are mechanically dimensioned such that the tensioning sleeve 1182 can be pushed on once in the thrust direction 1191, but, if an attempt is made to remove the tensioning sleeve 1182, the latching hooks 1179 break off from the connection piece 1061, thus preventing disassembly and repeat assembly. In an alternative embodiment, however, these can also be permanent, analogously to the alternatives described above. To make it easier to slide on, each latching hook 1179 can have a beveled surface (not shown) in the direction of the longitudinal axis 1192, such that each latching hook 1179 can slide onto the corresponding roof-shaped latching lug 1077 in the thrust direction 1191 and is then securely locked for an inverse movement. This makes it easier to push on the respective tensioning sleeve 1182 and makes removal even more difficult.

1. An endoscope system, in particular a disposable medical endoscope, comprising:
a handle housing for gripping and/or operating the endoscope system and/or for receiving functional components,
a shaft and a tip, wherein the shaft mechanically connects the handle housing to the tip along a longitudinal axis, and the tip has an image recording means for recording an image of a viewing region; and
the handle housing has a connection device with a clamping element or a plurality of clamping elements and a tensioning element, wherein the clamping element or the clamping elements allows or allow the shaft to be inserted into the connection device in a mounting position of the tensioning element, and the tensioning element moves the clamping element or the clamping elements mechanically to the shaft in a fastening position, in such a way that the shaft is received in a mechanically fixed manner on the handle housing by means of a force fit or form fit between the clamping element or clamping elements and the shaft.

2. The endoscope system as claimed in claim 1, wherein the clamping element or the clamping elements is or are arranged parallel or substantially parallel to the longitudinal axis, in particular distributed around the longitudinal axis.

3. The endoscope system as claimed in claim 1, wherein the clamping element or the clamping elements has or have a structuring facing the shaft, wherein in particular the shaft has a corresponding structuring facing the clamping element or clamping elements.

4. The endoscope system as claimed in claim 3, wherein the structuring and/or the corresponding structuring acts or act frictionally by means of the force fit between the clamping element or the clamping elements and the shaft, such that a coefficient of friction is increased and the shaft is thus mechanically fixed in the handle housing with a higher load capacity.

5. The endoscope system as claimed in claim 3, wherein the structuring and the corresponding structuring engage on the shaft, by means of respective structural elements and respective corresponding structural elements, in a form-fitting manner between the clamping element or the clamping elements and the shaft, wherein in particular structural elements and corresponding structural elements are arranged along the longitudinal axis in order to increase a transmissible torque between the shaft and the connection device and/or structural elements and corresponding structural elements are arranged along a circumference around the longitudinal axis in order to increase transmissible tensile forces.

6. The endoscope system as claimed in claim 1, wherein the tensioning element is annular and partially or completely surrounds the clamping element or clamping elements in the fastening position.

7. The endoscope system as claimed in claim 1, wherein the tensioning element has a latch or a plurality of latches, wherein the latch or the latches engages or engage in the fastening position in a corresponding latch receptacle or a respective corresponding latch receptacle on the connection device, wherein the latch or the latches are designed and mechanically weakened in particular by means of a weakening, in such a way that a movement of the tensioning element out of the fastening position causes the respective latch and/or the respective latch receptacle to be destroyed.

8. The endoscope system as claimed in claim 7, wherein the latch is designed to act axially along the longitudinal axis, such that the tensioning element is movable from the mounting position into the fastening position along the longitudinal axis and is fixed in a latching manner in the fastening position by means of the latch or the latches.

9. The endoscope system as claimed in claim 7, wherein the latch or the latches forms or form a bayonet lock, wherein the latch receptacle or the latch receptacles is or are designed in particular as latching lugs protruding from the connection device, and the latch or the latches is or are designed to be hook-shaped, such that the tensioning element can be moved along the longitudinal axis into the fastening position and can be fixed in the fastening position by means of a rotation about the longitudinal axis.

10. The endoscope system as set forth in claim 1, wherein the tensioning element can be screwed onto the connection means by means of a thread, such that the tensioning element is screwed on in the fastening position.

11. The endoscope system as set forth in claim 1, wherein the tensioning element, in particular on an accessible outer side facing away from the shaft, has a structure for applying forces for transferring the tensioning element into the fastening position, such that the forces for transferring the tensioning element into the fastening position can be applied manually by means of the structure and/or by means of a tool.

12. The endoscope system as claimed in claim 11, wherein the structure has depressions introduced radially into the tensioning element and/or elevations applied to the tensioning element and/or recesses for fingers of an operator and/or a corresponding tool which are introduced axially into the tensioning element or applied to the tensioning element, in particular surrounding the shaft.

13. The endoscope system as set forth in claim 1, wherein the connection means is designed as a separate transition piece between the handle housing and the shaft and in particular has an attachment, wherein the attachment is able to be brought into engagement with a corresponding receptacle on the handle housing.

14. The endoscope system as set forth in claim 1, wherein the connection means has a passage arranged orthogonally or substantially orthogonally to the longitudinal axis in order to facilitate mounting of the shaft on the handle housing, such that connections for the imaging means and/or mechanical control means guided within the shaft and protruding beyond one end of the shaft can be reached by means of a finger or several fingers of an operator.

15. The endoscope system as claimed in claim 13, wherein in that the handle housing has a first handle shell and a second handle shell, such that, when the first handle shell is joined to the second handle shell, the connection means designed as a transition piece is framed in a form-fitting manner between the first handle shell and the second handle shell by means of the attachment.
